(19) United States
(12) Patent Application Publication
(10) Pub. No.: US 2009/0104638 A1
(43) Pub. Date: Apr. 23, 2009

(54) Methods for Building Atomic Models of Protein Molecules and Determining Drug Candidates Using MGST1

(76) Inventors: Hans Hebert, Lidingo (SE); Ralf Morgenstern, Stockholm (SE)

Correspondence Address: PORTER WRIGHT MORRIS & ARTHUR, LLP;INTELLECTUAL PROPERTY GROUP, 41 SOUTH HIGH STREET, 28TH FLOOR, COLUMBUS, OH, 43215, US

(21) Appl. No.: 12/294,069

(22) PCT Filed: Mar. 22, 2007
(86) PCT No.: PCT/IB07/00824

§ 371 Date: Dec. 18, 2008

Related U.S. Application Data
(60) Provisional application No. 60/785,288, filed on Mar. 23, 2006.

(51) Int. Cl. C12Q 1/48; G06G 7/48

(52) U.S. Cl. 435/15; 703/11

(57) ABSTRACT

Methods for building an atomic model of a protein molecule comprising: (a) identifying a protein molecule with at least 20% sequence identity with Microsomal Glutathione Transferase 1 (MGST1) and (b) utilizing the atomic coordinates of MGST1 to obtain an atomic model of the identified protein molecule and methods for determining a drug candidate compound that interacts with members of the MAPEG superfamily, in particular MGST1 are also provided.

### FIELD OF THE INVENTION

The present invention relates to methods for building an atomic model of a protein molecule using Microsomal Glutathione Transferase 1 (MGST1) and methods for determining a drug candidate compound that interacts with proteins of the Membrane Associated Proteins in Eicosanoid and Glutathione Metabolism (MAPEG) superfamily, e.g., MGST1.

### SUMMARY OF THE INVENTION

The present invention provides methods for building an atomic model of a protein molecule. The methods comprise: (a) identifying a protein molecule with at least 20% sequence identity with Microsomal Glutathione Transferase 1 (MGST1) and (b) utilizing the atomic coordinates of MGST1 to obtain an atomic model of the identified protein molecule.

The present invention also provides methods for determining drug candidate compound. The methods comprise: A method for determining a drug candidate compound that interacts with Microsomal Glutathione Transferase 1 (MGST1) comprising: (a) identifying a drug candidate compound that interacts with MGST1 and (b) analyzing the interaction of the drug candidate compound with MGST1 or other members of the MAPEG protein superfamily.

### BRIEF DESCRIPTION OF THE FIGURES

The following Detailed Description maybe more fully understood in view of the drawings, in which:

FIG. 1. Experimental potential map and overall architecture of MGST1. A. Side view, cytoplasmic side up, in stereo through the transmembrane region showing TM2 in the centre flanked by TM1 from the same monomer to the left and the TM1 from the neighbouring subunit to the right. The refined model of the enzyme is superimposed onto the p6 map contoured at 1.2 σ. The proline residue at position 84 conserved among the MAPEG members gives rise to a kink of ˜15° of TM2. B. Side view, cytoplasmic side up, of the MGST1 monomer. The transmembrane helices have been labelled at their approximate N-terminal positions. C and D. The MGST1 trimer as viewed along the plane of the membrane (C) and from the cytoplasmic side (D). The approximate position of the hydrophobic core of the lipid bilayer has been depicted in the side view. In the top view, locations of the cytoplasmic E domains are indicated as contour plots at 1.0 σ and in corresponding colour. Domain E forms the connection between TM1 and TM2 and is in close contact with the loop between TM3 and TM4 of a neighbouring subunit. Residues H75 and E80 are involved in subunit interactions.

FIG. 2. Topology of MGST1 and polar interactions involving conserved residues in the MAPEG family. A. Schematic drawing of the topology of MGST1. B. Multiple sequence alignment of human MAPEG members and rat MGST1. Amino acid residues highlighted in yellow are conserved while blue indicates strict conservation. Numbering is according to the rat MGST1 sequence. h=helix, s=structure, l=loop, E=E domain c. Contacts between TM2 and TM3 through N77, N81, H116 and R113 in the upper part. Towards the lumenal face, side chains of phenylalanines 85, 106 and 109 are in close proximity.

FIG. 3. Catalytic site of MGST 1. A. 2Fₒ-F_{c}(blue) and Fₒ-F_{c}(green) maps calculated without GSH from p6 crystals visualised at 1.0 and 2.0 σ respectively with a unique position in the Fₒ-F_{c} map showing the strongest density distinctly away from the polypeptide trace and interpreted as a bound GSH molecule. The top view of the model shows the GSH positions relatively to the MGST1 trimer while the close-up in the right part shows that the GSH molecule is, apart from a close contact to R37, flanked by side chains from R72, R73, H75, E80, all of which are present in the cytoplasmic extension of TM2s from neighbouring subunits (a and b). This part of the sequence is also highly conserved in the MAPEG superfamily (see FIG. 2B). B. The central cavity in the MGST1 trimer as observed from calculations using the program HOLE . The large opening at the upper cytoplasmic side, is close to the GSH binding site. C. Side view of the cytoplasmic half of MGST1 trimer together with phospholipids at the approximate height of the lipid bilayer showing an opening in the structure towards the GSH site that provides a possible access point for hydrophobic

FIG. 4. Similarities between MGST1 and subunit 1 of cytochrome c oxidase. Structural comparison using DALI detected high scored similarity to subunit 1 of structurally investigated cytochrome c oxidases. Superimposition of the ba3 -cytochrome c oxidase (LEHK) (white) subunit onto the MGST1 model (yellow) by CE displays similarities of the transmembrane regions as well as in connectivities. The root mean square deviation between main chain atoms in the corresponding transmembrane parts of the two molecules is 2.04 A.

FIG. 5. Alignment between MGST1 and Dtdp-D-Glucose 4,6-Dehydratase (Rm1b) (PDB-id 1KEW) with the thymidine diphospate depicted in grey. The two strictly conserved residues in the MAPEG family N77 and R113 together with H116, conserved between MGST1 and MPGES1 (yellow backbone), are similarly arranged as corresponding residues in the dehydratase protein (white backbone). The substrate of the latter is depicted in grey. In a search for local structural patterns a significantly low E-value of 0.173 is estimated by PINTS.

FIG. 6. Close contacts of MGST1 trimers. In both the p6 (A, B) and the p22₁2₁ (C, D) 2D crystals the closest approach of MGST trimers is found between the symmetric tyrosine pairs Y115-Y145 and Y145-Y115 respectively.

FIG. 7. Catalytic mechanism involving three bound GSH molecules to the MGST1 homotrimer. The locations of the substrates relatively to the innermost TM2 helices of the enzyme form an annular interaction network. This provides a structural basis for third of the sites reactivity through tight binding simultaneously of only one GSH in the catalytically competent thiolate form. In addition, the on/off rate of thiolate formation, possibly modulated by the close proximity to the extension of TM1, the proline rich loop between TM3 and TM4 and domain E, may determine the dynamics of the active site.substrates.

### DETAILED DESCRIPTION OF THE INVENTION

Oxidative stress and exposure to toxic compounds are constant threats to living organisms. Efficient protection systems involving specific enzymes have emerged throughout evolution. Glutathione transferases, GSTs, are playing a crucial role in cellular biotransformation of electrophilic compounds through binding and positioning the tri-peptide glutathione (GSH), γ-L-glutamyl-L-cysteinyl-glycine, for nucleophilic attack. Distinct, ancient glutathione transferase protein families of cytoplasmic, microsomal, mitochondrial and bacterial origin have been identified based on sequence and structural similarity. In addition to the detoxification role played by GSTs, homologous members within the families may carry out distinctly different functions. Soluble GSTs from mammals, plants, bacteria and insects have been well characterized structurally and subdivided into several classes. The canonical cytosolic enzymes are dimers and related by evolution to glutaredoxin having the unique thioredoxin βαβαββα fold.

The inventors have determined the first detailed structure of a GST from the microsomal family, MGST1 (Microsomal Glutathione Transferase 1). Like most soluble GSTs, MGST1 catalyses conjugation of GSH to a number of electrophilic compounds and is therefore playing an important role in phase 2 biotransformation. In addition, MGST1 protects biological membranes from degradation through GSH dependent reduction of unspecifically peroxidised phospholipids. The microsomal GSTs, more recently termed MAPEG (Membrane Associated Proteins in Eicosanoid and Glutathione Metabolism) also contains members that are crucial for synthesis of mediators of fever, pain and inflammation. These pathophysiological responses are regulated by GSH dependent transformations of specific oxidised lipid intermediates, prostaglandin H₂₄ epoxide, to prostaglandin E₂ and leukotriene C₄ respectively. Thus, variations of a hitherto unknown common protein structure have emerged to selectively catalyse distinct activities with strong physiological and pathophysiological significance.

### Description of the MGST1 Protein Structure

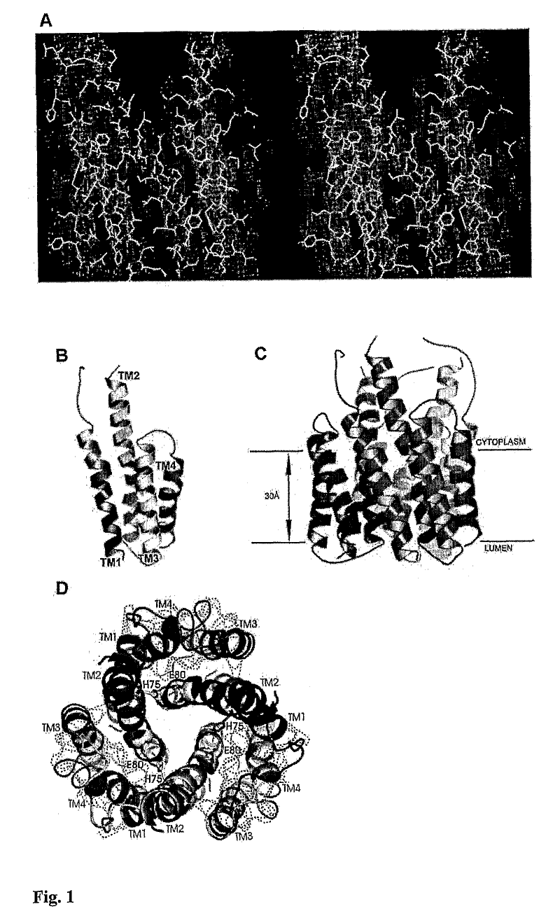

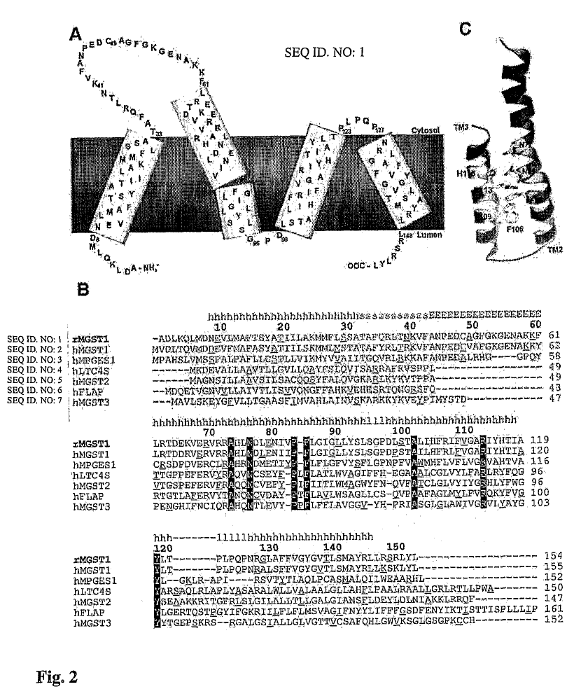

Upon reconstitution into lipid bilayers at low lipid/protein ratio MGST1 forms two-dimensional crystals of two different two-sided plane groups, p22₁2₁ and p6, both suitable for analysis by electron crystallography. The inventors have now solved the structure of the rat enzyme to 3.2 Å resolution using data from both crystal forms (FIG. 1A and Supplementary Table S1). As previously described both the p6 and the p22₁2₁ maps contain densities with three repeats of left-handed four-helix bundles making up a homotrimer known to be the functional unit (FIGS. 1B, C and D). In the present model of MGST1 the transmembrane helical segments with their predominantly cytoplasmic extensions are defined by residues N9-A32 (TM1), L62-S95 (TM2), L99-T122 (TM3), N128-L147 (TM4) respectively (FIGS. 1B and 2A). TM2 and TM3 are connected on the luminal side of the membrane by a short loop G96-D98, while aproline rich loop, P123-P127, on the other side connects TM3 and TM4. In the maps from both the p6 and the p22₁2₁ crystal form, distinct densities, henceforth referred to as domain E, are located between helices 1 and 2 of one subunit and 3 and 4 of the neighbouring subunit (FIG. 1D). Domain E is in direct contact with the end points of TM1 and TM2 and is thus forming the link between these two helices, i.e. T33-F61. Although the C-terminal end of domain E could not be modelled unambiguously it is clear that it extends towards the loop between TM3 and TM4 in the adjacent monomer indicating an important role in subunit interactions. As trypsin cleavage at residue K41 as well as covalent modification of C49 result in enzyme activation, it is suggested that release of constrained dynamics of the transmembrane helices through the interaction to the cytoplasmic domain E could be the molecular basis of the activation phenomenon. The profile of amide hydrogen/deuterium exchange along the polypeptide chain as observed by mass-spectrometry has been determined for MGST1. The alteration in exchange pattern following activation at the stress sensor C49 in domain E indicated a loosening of helix packing interactions which is consistent with our activation model. Other MAPEG members can not be activated by sulphydryl reagents and lack part of this region (FIG. 2B).

The present structure of MGST1 demonstrates distinct roles played by conserved amino acid residues among MAPEG members. Polar intrasubunit interactions facing the core of the monomer are important for TM2/TM3 stabilisation (FIG. 2C) involving residues N77, N81 from TM2 and R113, H116 from TM3. N77 and R113 are conserved among all MAPEG members (FIG. 2B). While not wishing to be bound by theory, an arginine or lysine side chain at position 113 seems to be important since the R113K mutant in MPGES I exhibits unaltered catalytic properties while R113 S decreases the activity. Interestingly N77, R113 and H116 form a triad of residues with a geometrical arrangement similar to that of Dtdp-D-Glucose 4,6-Dehydratase (Rm1b) (FIG. 5) where it plays a crucial role for positioning the substrate. The relative orientation between TM2 and TM3 is furthermore stabilised towards the luminal side of the membrane by π-π interactions involving phenylalanines in positions 85 (strictly conserved), 106 and 109 (FIG. 2C). Intersubunit dynamics have been implicated for the function and activation of MGST1 and are therefore of special interest. An intermolecular interaction between H75 and E80 (strictly conserved) in TM2 from two adjacent monomers is observed (FIG. 1D) and the mutations H75Q and E80Q results in decreased or abolished activity respectively.

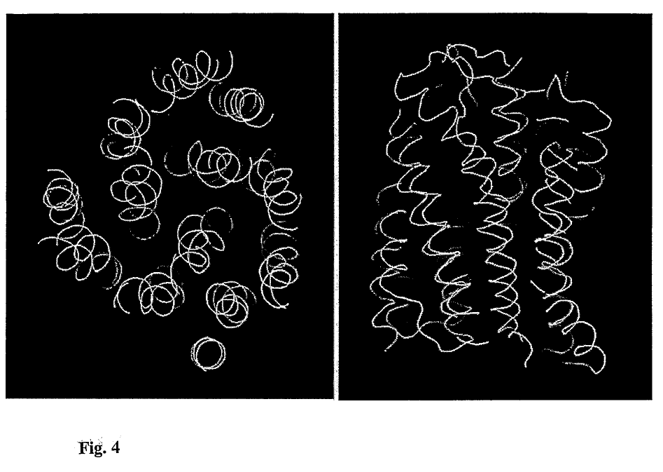

In order to explore similarities to other proteins, noted at lower resolution, the inventors performed structural comparison using DALI with both the MGST1 monomer and trimer as search models. A striking correspondence is found to subunit I of ba3-cytochrome c oxidase (FIG. 4) sharing no significant sequence similarity with MGST1. Not only the transmembrane regions were in agreement but also the connectivity between and order of α-helices. This observation indicates divergent evolution from a common structural ancestor. While not wishing to be bound by theory, the inventors speculate that when oxygen evolved in the atmosphere and oxidative stress became a threat, the precursor diverged into related functions comprising the reduction of toxic oxygen or oxidised lipid. This is in agreement with the general proposal that the present GSH conjugating capabilities of the GST families evolved from proteins with other functional properties.

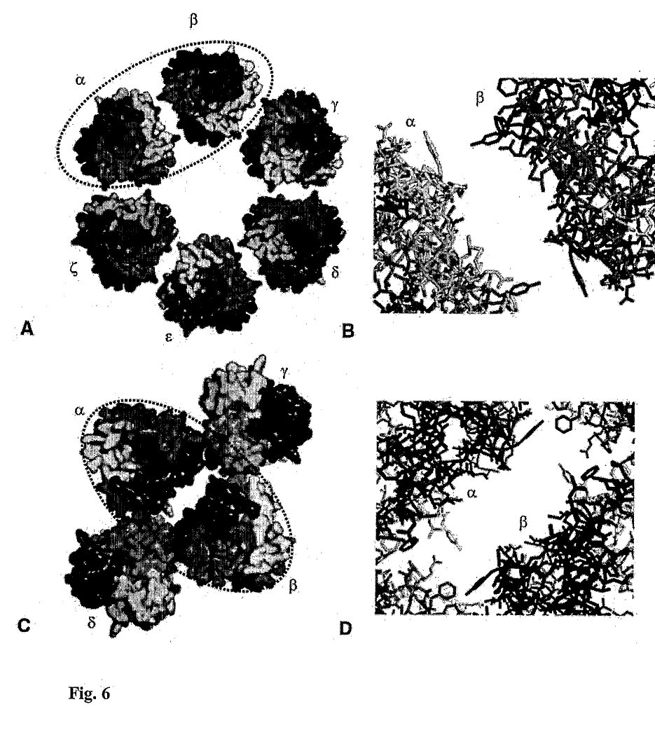

Two-dimensional crystallisation of MGST1 required a lower lipid to protein ratio as compared to other similarly grown 2D crystals of membrane proteins resulting in atomic models. Thus, it is expected that interaction between the hydrophobic transmembrane exposed belts of the protein would be tight. In fact, the locations of trimers in the two-dimensional crystals form topologically equivalent dimers of trimers (FIG. 6). It has been noted that MGST 1 can aggregate in a reversible manner which leads to fluorescence tag excimer formation consistent with a short intertrimer distance (<10Å) and in addition, interestingly, to enzyme activation.

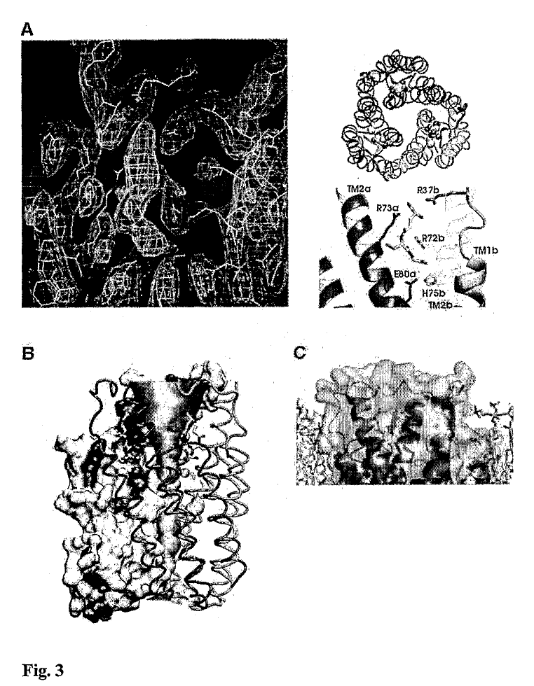

In the present study MGST1 is crystallised together with a saturating concentration of the thiol donor substrate (1 mM GSH). In accordance with recent nanospray mass spectrometry studies demonstrating three bound GSH molecules per trimer, the inventors observed a non-protein density close to the inner face of TM1, TM2 and domain E interpreted as the substrate molecule (FIG. 3A). Interactions of GSH with neighbouring monomers define a structural basis for functional subunit communication at the active site. The density suggests that GSH is bound in an extended conformation which is also observed in other GSH binding proteins. Although the resolution does not permit precise interactions to be modelled with certainty, the refined position and the predicted polar interactions are consistent with previous studies where GSH analogues were used to probe the enzyme active site preferences. For instance, a GSH analogue lacking the free γ-L-Glutamyl a-carboxyl group, that in the inventors' model forms a polar interaction to the guanidino group of R73, is not a substrate whereas removal of the adjacent α-amino group, showing no obvious binding interaction to the enzyme, leaves a functional substrate. R73 is preserved throughout the MAPEG superfamily except in FLAP which does not display catalytic activity. When R73 is subjected to mutational studies in LTC4S, replacement with lysine or histidine did not alter activity while replacement for threonine abolished function. Two additional arginines, R72 and R37, also point their side chains towards the GSH site in MGST1. Near the core of the structure, the previously discussed residues H75 and ESO from TM2 of two adjacent monomers (FIG. 1D) are also suggested to take part in GSH binding (FIG. 3A). While not wishing to be bound by theory, the inventors believe that the architecture of not only the complete oligomeric molecule but also of the active site is distinctly different from that of soluble glutathione binding proteins, including cytosolic GSTs, that contain the thioredoxin/glutaredoxin fold. Furthermore, the inventors' structure shows that the sequence segment 72 to 81 in MAPEG proteins, i.e. the cytoplasmic extension of TM2, plays a crucial role in stabilising the active site by fixation of the helical position and by defining a surface for substrate binding. In the MGST1 structure all non-hydrophobic amino acid residues in this stretch (R72, R73, H75, N77, D78, E80 and N81) do participate in these types of interactions. Moreover, three of the residues shared among all catalytic MAPEG proteins (R73, N77 and E80) are located in this region.

A fundamental aspect of glutathione transferase catalysis involves the stabilisation of the reactive nucleophilic thiolate anion form of GSH. The presence of the GSH thiolate has been demonstrated by spectroscopy in both soluble glutathione transferases and MGST1. In soluble glutathione transferases a tyrosine or serine hydroxyl has been shown to hydrogen bond to the GSH thiolate and thereby lower its pKa by several units to ≈6. In MGST1 GSH is surrounded by several residues that are potential hydrogen bond donors (H75, Y120, E80, R37/72/73). Since replacement of H75 and Y120 does not affect the activity to the extent predicted (and indeed observed) for a residue that is responsible for lowering the pKa of the GSH thiol, it may be concluded that MGST1 thiolate stabilisation operates by a novel interaction. While not wishing to be bound by theory, the inventors suggest that an arginine, most likely R72, fulfils this role in MGST1 and that the resulting unique charge compensation underlies the enzyme's ability to preferentially conjugate extremely hydrophobic substrates such as reactive chlorofluorocarbons.

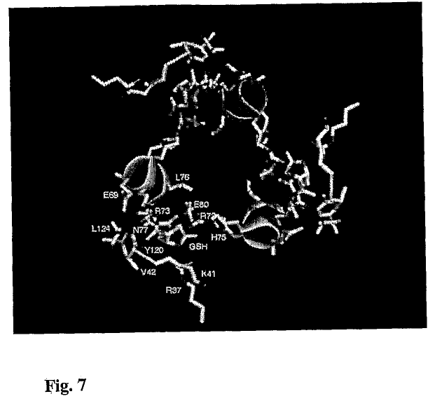

The inventors' structure including three GSH molecules bound to the MGST1 homotrimer supports a catalytic mechanism having third of the sites reactivity. The structural basis for active site communication is most likely the interlinked GSH coordination by residues in TM2 where each helix contributes both inter- and intrasubunit coordination (FIG. 7). The flexible nature of TM2, evidenced by high B-factors, allows heterogeneity and tight binding of only one GSH in the catalytically competent thiolate form. Once product is formed a neighbouring subunit can stabilise a GSH thiolate. In fact, while not wishing to be bound by theory, the inventors have observations suggesting that product bound in one subunit can augment thiolate formation in a neighbouring subunit and that thiolate formation, in turn, could facilitate product release. Thus the inventors suggest that the biological rationale for third of the sites reactivity is the necessity to prevent product inhibition. Another interesting possibility arising from this type of mechanism is dynamic active site scanning where the thiolate is rapidly forming and unforming on the three subunits efficiently sampling the presence of reactive substrates. Certainly H/D exchange upon GSH binding, although generally decreasing as expected, also show localised increases.

Most of the relatively few membrane protein structures known are responsible for selective, active or passive translocation across membranes of cargo reaching in size from ions to proteins. In comparison, the primary function of MGST1 is to catalyse chemical reactions with implications regarding approach of the hydrophilic GSH molecule and hydrophobic second substrates. Like oligomeric transporting membrane proteins MGST 1 has a central cavity, here facing the cytosol, but it rapidly narrows in the centre of the trimer making it impermeable to water or any larger molecule (FIG. 3B). While not wishing to be bound by theory, the inventors suggest that the exposed central surface forms binding sites for hydrophobic second substrates allowing for the observed variety in size and shape. The three GSH molecules in the wall of this cavity lends strong support to this proposal. In this model second substrates would have to enter the active site from the cytosol via the upper parts of the central cavity, a notion that is consistent with kinetic data. The localisation of positive charges in this otherwise hydrophobic region is also compatible with the strong binding/inhibitory properties of organic anions. The question then arises how do extremely hydrophobic substrates (e.g. phospholipid hydroperoxides for the glutathione peroxidase activity) enter the active site from the membrane. The inventors' structural model identifies an alternate access point to GSH at the subunit interface via a large opening facing the phospholipid headgroups at the cytosolic side of the membrane (FIG. 3C). Hence, while not wishing to be bound by theory, the inventors suggest the possibility of two electrophilic substrate binding sites and that access from the membrane via the phospholipid headgroups can be advantageous in catalytic conversion of extremely hydrophobic substrates. A corresponding but highly specific binding site for PGH could thus also be present in MPGES1. Interestingly, the substrate for MPGES1 is synthesised in the lumen of the endoplasmatic reticulum whereas the present work strongly suggests that the active site is located on the cytosolic side also for that protein. Even if it can not be excluded that MPGES1 could provide a central channel for PGH₂ translocation it seems unlikely since corresponding residues facing the centre of the MPGES1 trimer are bulky and hydrophobic.

Projection maps of two other MAPEG members, human MPGES1 and LTC4S, show striking similarity to MGST1. Thus it is likely that the structural principles for catalysis as displayed by MGST1, being completely different from that of functionally similar soluble proteins and thus representing a novel structural solution, will be representative for the MAPEG superfamily. In view of the 37% sequence identity between MGST1 and MPGES1 a homology model for these two proteins is expected to be particularly revealing. Thus the principal elements contributing to the difference in mechanism and substrate specificity including the broad substrate acceptance of MGST1 versus the selectivity of MPGES1 for PGH₂ may now be identified through complementary investigations.

In summary, while not wishing to be bound by theory, the inventors propose a novel catalytic mechanism involving subunit interactions that takes advantage of the membrane location of MGST1 allowing entry of hydrophobic substrates to the active site both from the cytosol and the phospholid bilayer. The latter entry point is catering for extremely hydrophobic molecules that hardly leave the membrane. In addition, by utilising charge compensation for thiolate anion stabilisation the enzyme allows small extremely hydrophobic substrates to approach the thiolate much more efficiently.

By determining the atomic structure of a key enzyme or the MAPEG superfamily, MGST1 (Microsomal Glutathione Transferase 1), the inventors are able to determine the specificity and function of these proteins that are potential targets for treatment of common diseases such as rheumatoid arthritis and asthma, the results should impact on drug development.

Accordingly, the inventors have invented methods for building an atomic model of a protein molecule comprising: (a) identifying a protein molecule with at least 20% sequence identity with Microsomal Glutathione Transferase 1 (MGST1) and (b) utilizing the atomic coordinates of MGST1 to obtain an atomic model of the identified protein molecule. In one embodiment, the protein molecule is a MAPEG protein molecule. In another embodiment, the protein molecule has at least 30% sequence identity with MGST1. One skilled in the art will appreciate the various models and candidates that may be produced by knowing the atomic coordinates of one protein. In one embodiment, the atomic model comprises a homology model, which is obtained using a modelling software program. In another embodiment, the atomic model comprises an experimental model, which is obtained with molecular replacement.

Once a protein molecule has been identified, a drug candidate compound that interacts with the identified protein molecule or with MGST1 may be determined. In one embodiment, a drug candidate compound is identified by using the atomic structure of the identified protein molecule to design a drug candidate compound. In another embodiment, the drug candidate compound is identified by (a) contacting the drug candidate compound with the identified protein molecule; and (b) measuring for a change in the expression or activity of the identified protein molecule.

One skilled in the art will appreciate the various means in which a drug candidate compound interacts with a protein molecule. In one embodiment, a drug candidate compound that decreases the expression or activity of the protein molecule indicates that the compound is an inhibitor of the protein molecule. hi another embodiment, a compound that increases the expression or activity of the protein molecule indicates that the compound is a promoter of the protein molecule. One skilled in the art will appreciate the various methods for contacting the drug candidate compound with a protein molecule, any of which may be employed herein. One skilled in the art will also appreciate the various methods for measuring a change in the expression or activity of the protein, any of which may be employed herein.

The interaction of the drug candidate compound with the identified protein molecule may be analyzed. In one embodiment, the interaction of the drug candidate compound with the active site of the identified protein molecule is analyzed with a docking-program. In addition, the structure of the interaction of the candidate compound with the protein molecule may be obtained using molecular replacement.

Furthermore, one skilled in the art will appreciate that in order to comprehensively determine a suitable drug candidate compound that interacts with the protein molecule at least one catalytic position of the protein molecule may be mutated prior to contacting the drug candidate compound with the protein molecule. In one embodiment, the mutation comprises a substitution of at least one amino acid. In another embodiment, the mutation comprises a deletion of at least one amino acid.

### Materials and Methods

Specimen preparation. MGST1 is purified from rat liver microsomes and two-dimensional crystals with p6 and p22₁2₁ two-dimensional plane group symmetry are prepared by reconstitution into phospholipid bilayers at molar lipid to protein ratios varying between 3 and 5 as previously described. The quality of the reconstituted crystals are evaluated by electron microscopy of negatively stained specimens. Excellent crystal preparations are selected for cryo-electron microscopy. These specimens are prepared on carbon coated molybdenum grids and embedded in 3-7% (w/v) trehalose using the back injection technique. Specimens selected for high-tilt data collection are prepared by the carbon sandwich technique.

Data collection. Specimens of both crystal forms are imaged at tilt angles ranging from 0° to 62.9° at 4K specimen temperature on Kodak SO-163 film using a JEOL3000SFF electron microscope equipped with a liquid He cooled stage and a field-emission gun operated at an acceleration voltage of 300 kV. The images are subsequently developed for 12 minutes in full-strength D19 developer. Electron diffraction patterns are recorded using a FEI CM120 electron microscope equipped with a TVIPS 1k×1k, and the JEOL3000SFF initially fitted with a Gatan 2k×2k and later with a Gatan 4k×4k slow-scan CCD camera respectively.

Data processing. Image negatives are evaluated by optical diffraction and scanned using a Zeiss SCAI scanner with a pixel size of 7 μm. Several rounds of computational unbending and correction for the contrast transfer function (CTF) are performed by the MRC program system (6) to produce initial 3D data sets for the different crystal forms. Tilt-angles and CTFs accurately determined during the first processing round are used for reprocessing all good images in a second round of unbending using the MAKETRAN program procedure. Final image amplitudes and phase values are extracted following tilted CTF and beam tilt correction. Diffraction amplitudes collected on the three different CCD cameras are processed using the MRC electron diffraction software. Image phases and electron diffraction amplitudes are merged using the LATLINE program from the MRC suite.

Map calculation. Maps are calculated from reflection files containing data to 3.04 Å (p6) and 3.09 Å (p22₁2₁) using CCP4 software. To evaluate the degree of non-crystallographic symmetry (ncs) in the p22₁2₁ crystal form, monomers from the unsymmetrised map are correlated. The correlation coefficients at 120° and 240° rotations are 0.82 demonstrating a high similarity between the subunits and thus the ncs in the centre of the trimer are applied. To quantify correspondence between the maps from the hexagonal and orthorhombic crystal symmetries, trimers from each map are aligned and correlated resulting in a maximum overall correlation coefficient of 0.84. The high degree of correspondence allowed calculation of a cross crystal averaged trimer containing data from both crystal forms.

Model building. Model building is performed in O initially using a combination of the three calculated maps that are skeletonised and fitted with secondary structure poly-alanine templates. Subsequently, mutations to the appropriate amino acids are performed using identifiable side chains. Residues N9 to F43 and L62 to L147 making up 78.6% of the polypeptide, could be built corresponding to all of the trans-membrane parts of the helices and to a large extent also extra-membranous regions.

Refinement and rebuilding. Given its higher symmetry and completeness the p6 crystal data set are used for refinement of the model while the p22₁2₁ data are used as an independent test and quality control data set. The initial monomer model are translated and rotated into the p6 map. Symmetry related monomers are generated in the p6 map in O rendering a trimer that are transferred and rebuilt into the appropriate position in the p22₁2₁ map. Numerous cycles of rigid body and restrained refinement using very tight geometry restraints of the model are performed against the p6 electron diffraction data set truncated at 3.2 Å using REFMAC5 including geometry idealisation and manual rebuilding in O. At the present resolution the difference between using electron- or X-ray form factors are negligible, thus X-ray values are used throughout the refinement. 2Fₒ-F_{c} and Fₒ-F_{c} maps are continuously generated to evaluate the accuracy and quality of the refined model although the experimental map are used for all rebuilding steps in order to avoid model bias. Reliable R_{free} values are obtained by using a final fraction of 9.4% of the observed structure factor amplitudes. In addition to the conventional and free R-factors the refinement process are monitored by comparing the p22₁2₁ Fₒ- and F_{c}-values. This residual denoted R are calculated as the conventional R-value between observed p22₁2₁ amplitudes and corresponding calculated amplitudes from the model following translation and alignment of the trimer to the orthorhombic unit cell without any further refinement. The initial Rₒᵣₜ are close to 60% before refinement. PROCHECK are used to examine the molecular geometry.

Structural alignment. For structural comparison the monomer and trimer are submitted to the DALI/FSSP server. The top seven hits, all having a Z-score above 5.0 are selected for structural alignment using CE.

TABLE 1
Data collection and refinement statistics

| Two-dimensional crystal parameters | | |
|---|---|---|
| Two-sided plane-group | p22₁2₁ | p6 |
| Unit cell (Å) | a = 91.9 | a = 81.8 |
| | b = 90.8 | b = 81.8 |
| | c = 100.0* | c = 100.0* |
| | γ = 90.0° | γ = 120.0° |
| Phase determination from images | | |
| Number of images used | 77 | 53 |
| Maximum tilt angle (°) | 62.8 | 62.9 |
| Resolution in and normal to | 3.5/7.0 | 3.5/7.0 |
| membrane plane (Å)** | | |
| No. of observed/used phases | 41132/9561 | 17915/5300 |
| Fourier space sampled (%) | 73.4 | 74.4 |
| Phase residual, overall/4.0-3.5 Å | 21.6/54.6 | 30.8/42.4 |
| resolution shell (°) | | |
| Amplitude determination from | | |
| electron diffraction | | |
| No. of diffraction patterns | 44 | 120 |
| Maximum tilt angle (°) | 60.6 | 62.6 |
| Resolution in and normal to | 3.1/4.5 | 3.0/4.0 |
| membrane plane (Å)** | | |
| No. of observed/used amplitudes | 29211/11073 | 51754/5154 |
| Fourier space sampled, overall/ | 58.3 | 78.3/64.3 |
| 3.35-3.16 Å (p6) (%) | | |
| I/σ, overall/4.0-3.5 Å | 6.0/2.5 | 12.1/6.0 |
| R_{Friedel} (%) | 24.9 | 12.7 |
| R_{merge} (%) | 34.7 | 28.8 |
| Crystallographic refinement | | |
| Resolution (Å) | | 10.0-3.2 |
| No. of reflections | | 4409 |
| No. of atoms, protein/substrate | | 964/20 |
| R_{work} (%) | | 33.9 |
| R_{Free} (%) | | 37.6 |
| Rₒᵣₜ*** (%) | | 49.1 |
| Overall B-factor | | 26.7 |
| R.m.s. deviations | | |
| Bond lengths (Å) | | 0.013 |
| Bond angles (°) | | 1.863 |
| Ramachandran plot distribution (%) | | 60.0/38.1/ |
| | | 1.9/0.0 |

*Assumed for sampling along z*. For lattice line adaption a thickness of 65 Å is used.
**From calculation of point spread function.
***Calculated as conventional R-value between observed p22₁2₁ amplitudes and corresponding calculated amplitudes from model following alignment of the trimer in the unit cell without any further refinement. The starting value before refinement are close to 60%.

What is claimed is:

1. A method for building an atomic model of a protein molecule comprising: (a) identifying a protein molecule with at least 20% sequence identity with Microsomal Glutathione Transferase 1 (MGST1) and (b) utilizing the atomic coordinates of MGST1 to obtain an atomic model of the identified protein molecule.

2. The method of claim 1, wherein the protein molecule is a Membrane Associated Protein in Eicosanoid and Glutathione Metabolism (MAPEG) protein molecule.

3. The method of claim 1, wherein the atomic model comprises a homology model.

4. The method of claim 3, wherein the homology model is obtained by a modelling software program.

5. The method of claim 1, wherein the atomic model comprises an experimental model.

6. The method of claim 5, wherein the experimental model is obtained with molecular replacement.

7. The method of claim 1, further comprising (c) identifying a drug candidate compound that interacts with the identified protein molecule, wherein the atomic structure of the identified protein molecule is used to identify a drug candidate compound.

8. The method of claim 7, further comprising (d) analyzing the interaction of the drug candidate compound with the identified protein molecule.

9. The method of claim 8, wherein the interaction of the drug candidate compound with the active site of the identified protein molecule is analyzed with a docking-program.

10. The method of claim 8, wherein the structure of the interaction of the drug candidate compound with the identified protein molecule is obtained using molecular replacement.

11. The method of claim 1, further comprising (c) identifying a drug candidate compound that interacts with the identified protein molecule by (1) contacting the drug candidate compound with the identified protein molecule and (2) measuring for a change in the expression or activity of the identified protein molecule.

12. The method of claim 11, wherein a drug candidate compound that decreases the expression or activity of the identified protein molecule indicates that the drug candidate compound is an inhibitor of the identified protein molecule.

13. The method of claim 11, wherein a drug candidate compound that increases the expression or activity of the identified protein molecule indicates that the drug candidate compound is a promoter of the identified protein molecule.

14. The method of claim 6, wherein at least one catalytic position of the identified protein molecule is mutated prior to identifying a drug candidate compound that interacts with the identified protein molecule.

15. The method of claim 14, wherein the mutation comprises a substitution of at least one amino acid.

16. The method of claim 14, wherein the mutation comprise a deletion of at least one amino acid.

17. A method for determining a drug candidate compound that interacts with Microsomal Glutathione Transferase 1 (MGST1) comprising: (a) identifying a drug candidate compound that interacts with MGST1 and (b) analyzing the interaction of the drug candidate compound with MGST1.

18. The method of claim 17, wherein the interaction of the drug candidate compound with the active site of the MGST1 is analyzed with a docking-program.

19. The method of claim 17, wherein the structure of the interaction of the drug candidate compound with MGST1 is obtained using molecular replacement.

20. The method of claim 17, wherein the drug candidate compound is identified by using the atomic structure of MGST1 to design a drug candidate compound.

21. The method of claim 17, wherein the drug candidate compound is identified by (a) contacting the drug candidate compound with MGST1; and (b) measuring for a change in the expression or activity of the protein molecule.

22. The method of claim 21, wherein a drug candidate compound that decreases the expression or activity of MGST1 indicates that the drug candidate compound is an inhibitor of MGST1.

23. The method of claim 21, wherein a drug candidate compound that increases the expression or activity of MGST1 indicates that the drug candidate compound is a promoter of MGST 1.

24. The method of claim 17, wherein at least one catalytic position of MGST1 is mutated prior to identifying a drug candidate compound that interacts with MGST1.

25. The method of claim 24, wherein the mutation comprises a substitution of at least one amino acid.

26. The method of claim 24, wherein the mutation comprise a deletion of at least one amino acid.


SEQUENCE LISTING (7 sequences)

SEQ ID NO: 1 - protein, 154 aa, Artificial Sequence
  rat Microsomal Glutathione Transferase 1 (rMGST1)
ADLKQLMDNEVLMAFTSYATIILAKMMFLSSATAFQRLTNKVFANPEDCAGFGKGENAKKFLRTDEKVERVRRAHLNDLENIVPFLGIGLLYSLSGPDLSTALIHFRIFVGARIYHTIAYLTPLPQPNRGLAFFVGYGVTLSMAYRLLRSRLYL

SEQ ID NO: 2 - protein, 155 aa, Artificial Sequence
  human Microsomal Glutathione Transferase 1 (hMGST1)
MVDLTQVMDDEVEMAFASYATIILSKMMLMSTATAFYRLTRKVFANPEDCVAFGKGENAKKYLRTDDRVERVRRAHLNDLENIIPFLGIGLLYSLSGPDPSTAILHFRLFVGARIYHTIAYLTPLPQPNRALSFFVGYGVTLSMAYRLLKSKLYL

SEQ ID NO: 3 - protein, 152 aa, Artificial Sequence
  human Microsomal Prostaglandin E Synthase 1 (hMPGES1)
MPAHSLVMSSPALPAFLLCSTLLVIKMYVVAIITGQVRLRKKAFANPEDALRHGGPQYCRSDPDVERCLRAHRNDMETIYPFLFLGFVYSFLGPNPFVAWMHFLVFLVGRVAHTVAYLGKLRAPIRSVTYTLAQLPCASMALQILWEAARHL

SEQ ID NO: 4 - protein, 150 aa, Artificial Sequence
  human Leukotriene C4 synthase (hLTC4S)
MKDEVALLAAVTLLGVLLQAYFSLQVISARRAFRVSPPLTTGPPEFERVYRAQVNCSEYFPLFLATLWVAGIFFHEGAAALCGLVYLFARLRYFQGYARSAQLRLAPLYASARALWLLVALAALGLLAHFLPAALRAALLGRLRTLLPWA

SEQ ID NO: 5 - protein, 147 aa, Artificial Sequence
  human Microsomal Glutathione Transferase 2 (hMGST2)
MAGNSILLAAVSILSACQQSYFALQVGKARLKYKVTPPAVTGSPEFERVFRAQQNCVEFYPIFIITLWMAGWYFNQVFATCLGLVYIYGRHLYFWGYSEAAKKRITGFRLSLGILALLTLLGALGIANSFLDEYLDLNIAKKLRRQF

SEQ ID NO: 6 - protein, 161 aa, Artificial Sequence
  human 5-Lipoxygenase-Activating Protein (hFLAP)
MDQETVGNVVLLAIVTLTSVVQNGFFAHKVEHESRTQNGRSFQRTGTLAFERVYTANQNCVDAYPTFLAVLWSAGLLCSQVPAAFAGLMYLFVRQKYFVGYLGERTQSTPGYIFGKRIILFLFLMSVAGIFNYYLIFFFGSDFENYIKTISTTISPLLLIP

SEQ ID NO: 7 - protein, 152 aa, Artificial Sequence
  human Microsomal Glutathione Transferase 3 (hMGST3)
MAVLSKEYGFVLLTGAASFIMVAHLAINVSKARKKYKVEYPTMYSTDPENGHIFNCIQRAHQNTLEVYPPFLFFLAVGGVYHPRIASGLGLAWIVGRVLYAYGYYTGEPSKRSRGALGSIALLGLVGTTVCSAFQHLGWVKSGLGSGPKCCH